(19) United States
(12) Patent Application Publication
(10) Pub. No.: US 2007/0048393 A1
(43) Pub. Date: Mar. 1, 2007

(54) Topical turmeric skin care products

(75) Inventor: Alexander A. Bommarito, Freeland, MI (US)

Correspondence Address: McCulloch PLC, 1st Floor, 5291 Colony Drive North, Saginaw, MI, 48638, US

(73) Assignee: SPRAY TANNING, INC.

(21) Appl. No.: 11/509,212

(22) Filed: Aug. 24, 2006

Related U.S. Application Data
(60) Provisional application No. 60/713,560, filed on Sep. 1, 2005.

(51) Int. Cl. A61K 36/906; A61K 36/752; A61K 36/45

(52) U.S. Cl. 424/732; 424/736; 424/756

(57) ABSTRACT

A topical skin care product containing therapeutic concentrations of turmeric and other constituents which enable the product to be used for cosmetic, protective, and healing purposes without the yellow staining of skin or clothing.

### CROSS REFERENCE TO RELATED APPLICATION

This application claims the benefit of the filing date of U.S. Provisional Application No. 60/713,560 filed Sep. 1, 2005.

This invention relates to a topical skin care product containing turmeric, or extracts thereof, such as curcumin, curcuminoids, and which may be applied to a person's skin without staining the skin yellow.

### BACKGROUND OF THE INVENTION

Turmeric and its ingredients have been used for thousands of years because of the therapeutic properties thereof. Topical use of turmeric-containing compounds has been limited because the turmeric colors the skin yellow. Yellowish skin is simply not acceptable to many people regardless of the therapeutic benefits which may be associated with turmeric. In addition, the application of a substance containing turmeric in many instances causes yellow staining of a person's clothing, a result which also is unacceptable. A topical skin care product formulated in accordance with the invention overcomes these objectionable characteristics while retaining the protective and healing properties of turmeric.

### SUMMARY OF THE INVENTION

Natural medicines, such as turmeric, have been used topically for the avoidance and treatment of many conditions. For example, turmeric is used in many areas of the world for the treatment of wounds, burns, including sun burns, and psoriasis, as well as in body lotions, moisturizers, antiseptic agents, beauty aids, allergic reaction formulations, anti-inflammatory products, anti-cancer products, anti-aging products, anti-oxidant products, and osteoporosis products, including vitamin D.

Although the therapeutic properties of turmeric are well known, its use as a topical agent is limited because it turns the skin yellow and stains clothing. The yellowish appearance of skin induced by the use of turmeric may last for several days, whereas the yellow staining of clothing may be permanent.

Since the incorporation of turmeric in topical formulations results in yellowing of the skin and since yellowing of the skin is objectionable to many persons, topical applications of turmeric are limited. It is possible for a person to ingest turmeric, but adequate skin treatment is difficult to achieve by oral administration of turmeric. A substance formulated and applied in accordance with the invention overcomes these objectionable characteristics and provides for the incorporation of turmeric into topical skin care products including solutions, lotions, creams, and ointments.

### THE PREFERRED EMBODIMENT

The invention makes possible the incorporation of turmeric and its active ingredients into topical skin care creams, lotions, ointments, and sprayable solutions without turning the skin yellow or staining clothing worn by the person to whom the substance is applied. Turmeric is commercially available as a dry powder having the standard characteristics known in pharmacognosy. Since turmeric can vary in its concentration of active ingredients due to growing conditions and harvesting techniques, a known source and standard of turmeric can and should be used. A suitable powder source is McCormick & Co., among others, and a suitable source of turmeric extract is Camden Gray Company, among others.

In the preparation of the topical skin care product according to the invention a quantity of turmeric powder is weighed and a known weight of such powder is added to a solvent system for extraction of the active ingredients (curcumin, curcuminoids) of the turmeric. Appropriate solvent systems are well known and a good review thereof is available in the Journal of Agriculture and Food Chemistry (2003). A typical procedure combines 100 cc of ethanol with 100 cc of isopropyl alcohol and 100 cc of acetone with 30 grams of turmeric powder. However, other procedures and concentrations may be used.

The active ingredients of turmeric are in the soluble liquid portion of the mixture. If allowed to dry, two distinct layers will be formed. The top layer will include the color red mixed with the characteristic yellow color. In the procedure of this invention the liquid phase containing the many active ingredients of turmeric are used. They are added to a mixture of blueberries, coffee berries or beans, green tea extract, pomegranate extract or powder formulated to receive the turmeric and eliminate or mask its characteristic yellow color.

In the preparation of the blueberry-turmeric mixture, for example, the blueberries or skins thereof are broken and mixed well with lemon or other citrus juice in about equal parts. The mixture is heated to a level below the boiling temperature of the mixture until the ingredients are thoroughly mixed. The heating step assists in the proper color development or biochemical extraction of the active phytochemicals. Following the heating step the liquid phase of the mixture is separated from the solid phase by allowing the mixture to stand, or by filtration. The liquid phase then is combined with the turmeric extract at the desired concentration. The filtrate may be at ambient or a somewhat higher temperature as a result of heating. Acceptable concentrations range from about 0.5 cc to 4.0 cc of turmeric extract to 10 cc blueberry lemon juice mixture. The resulting solution provides an adequate therapeutic concentration of the turmeric active ingredients for topical use without the yellow staining characteristics.

If desired, the solution containing the turmeric extract and blueberry lemon juice mixture may be incorporated into an ointment base which accepts a liquid solution. For example, 10 grams of an ointment base, such as a hydrophilic absorption base available under the trademark AQUAPHOR from Biersdof, Inc., will accept an equal amount of fluid or approximately 10 cc of the above mixture. Instead of incorporating the active ingredients and blueberry lemon mixture in an ointment base, it can be added to other topical lotions, creams, or sprayable solutions, including moisturizers.

A mixture containing the other ingredients referred to above may be prepared in substantially the same way. Such other ingredients also may be included with the blueberry-based substance in desired proportions if an increase in the concentration of turmeric is desired.

The combining of the ingredients is easily accomplished in known ways by persons skilled in the art.

If the turmeric active ingredients referred to above are combined only with common body solutions, lotions, ointments, or the like, the resultant substance will have a yellow appearance and, if applied to a person's skin, will stain the skin yellow. However, combining the same concentration of the turmeric active ingredients with the blueberry lemon juice mixture and then incorporating the combination in a solution, lotion, ointment, or the like results in a product having a blue, tan or brown appearance according to the concentration of the turmeric. The resulting product does not possess the staining properties common to blueberries or turmeric or the active ingredients thereof.

Although blueberries and lemon juice are preferred, it is possible to use huckleberries, coffee beans, green tea extract, pomegranate, bilberries, bearberries, moonseed berries, mountain currant (ribes novadense), and blue/purple grapes of many varieties, in lieu of the blueberries. Instead of lemon juice other citrus juices may be used. Other coloring, natural or synthetic, also may be used without departing from the scope of the invention.

This disclosure is representative of a presently preferred embodiment of the invention, but is intended to be illustrative rather than definitive thereof. The invention is defined in the claims.

I claim:

1. A topical composition applicable to a person's skin comprising a therapeutic mixture of turmeric having a characteristic yellow color capable of staining a person's skin yellow and a substance which reacts with said turmeric in such manner as to prevent yellow staining of the person's skin.

2. The composition according to claim 1 wherein said substance includes the juice of blueberries.

3. The composition according to claim 1 wherein said substance includes citrus juice.

4. The composition according to claim 3 wherein said citrus juice is lemon juice.

5. The composition according to claim 1 wherein in the formation of said substance said mixture is heated to a temperature less than the boiling temperature of said mixture.

6. The composition according to claim 1 wherein said mixture has a concentration of between about 0.5 cc to 4.0 cc of turmeric and about 10 cc of said substance.

7. The composition according to claim 1 wherein said mixture is incorporated in an ointment.

8. The composition according to claim 1 wherein said mixture is incorporated in a lotion.

9. The composition according to claim 1 wherein said mixture is incorporated in a cream.

10. The composition according to claim 1 wherein said mixture is incorporated in a sprayable solution.

11. The composition according to claim 1 wherein said mixture is incorporated in a moisturizer.

12. The composition according to claim 1 wherein said mixture is incorporated in an antiseptic agent.

13. The composition according to claim 1 wherein said mixture is incorporated in a beauty aid.

14. The composition according to claim 1 wherein said mixture is incorporated in a wound healing product.

15. The composition according to claim 1 wherein said mixture is incorporated in an allergy relief product.

16. The composition according to claim 1 wherein said mixture is incorporated in an anti-inflammatory product.

17. The composition according to claim 1 wherein said mixture is incorporated in an anti-cancer product.

18. The composition according to claim 1 wherein said mixture is incorporated in a sunscreen product.

19. The composition according to claim 1 wherein said mixture is incorporated in an anti-aging product.

20. The composition according to claim 1 wherein said mixture is incorporated in an anti-oxidant product.

21. The composition according to claim 1 wherein said mixture is incorporated in an osteoporosis product.

22. The composition according to claim 21 wherein said osteoporosis product contains vitamin D.

23. The composition according to claim 1 wherein said substance includes citrus juice and the juice of blueberries.

24. The composition according to claim 23 wherein said mixture is incorporated in a flowable product.
